(19) United States
(12) Patent Application Publication
(10) Pub. No.: US 2013/0189336 A1
(43) Pub. Date: Jul. 25, 2013

(54) Cosmetic Compositions Including Ivy Derived Nanoparticles

(71) Applicants: Mingjun Zhang, Palo Alto, CA (US); Lijin Xia, Knoxville, TN (US); Scott Lenaghan, Knoxville, TN (US)

(72) Inventors: Mingjun Zhang, Palo Alto, CA (US); Lijin Xia, Knoxville, TN (US); Scott Lenaghan, Knoxville, TN (US)

(73) Assignee: UNIVERSITY OF TENNESSEE RESEARCH FOUNDATION, Knoxville, TN (US)

(21) Appl. No.: 13/877,500

(22) PCT Filed: Oct. 5, 2010
(86) PCT No.: PCT/US2010/051468

§ 371 Date: Apr. 3, 2013

(51) Int. Cl. A61K 8/97; A61Q 17/04

(52) U.S. Cl. CPC A61K 8/97; A61Q 17/04
USPC 424/401; 424/59

(57) ABSTRACT

Ivy derived nanoparticles and methods for their preparation and use are described herein. Further described herein are cosmetic compositions containing the ivy derived nanoparticles and methods for their use as sunscreens.

### STATEMENT REGARDING FEDERALLY FUNDED RESEARCH

This invention was made with government support under Grant No. W911NF-10-1-0114 awarded by the United States Army Research Office. The government has certain rights in the invention.

### FIELD

The subject matter disclosed herein generally relates to ivy derived nanoparticles and to methods of preparing cosmetic formulations including these nanoparticles. Also, the subject matter disclosed herein generally relates to methods of using the compositions described herein as sunscreens.

### BACKGROUND

Ultraviolet (UV) radiation is highly energetic electromagnetic radiation from light waves below that of the visible light spectrum. The wavelengths for UV radiation range from 100-400 nm, including UV-A (315-400 nm), UV-B (280-315 nm), and UV-C (100-280 nm) (see Diffey, B. L., Phys Med Biol 36:299-328 (1991)). While the earth's ozone layer blocks 98.7% of UV radiation from penetrating through the atmosphere, a small percentage of UV, comprising UV-A and some UV-B, can still reach the planet, which can cause harmful effects to humans (Pathak et al., Resonance, 7:71-80 (2002)). UV-C does not typically reach the surface of the planet, but due to its ability to cause DNA damage it is often used as a model for UV study in the laboratory. Depending on the time of exposure to sunlight, harmful UV-A/UV-B effects can include immediate distresses like blistering sunburns, and long term problems such as skin cancer, melanoma, cataracts, and immune suppression (Hockberger, P. E., Photochem Photobiol 76:561-579 (2002) and Longstreth et al., J Photochem Photobiol B, 46:20-39 (1998)). The underlying mechanism for UV-A damage involves oxidative stress and protein denaturation, while short wavelength UV-B radiation causes predominantly DNA damage in the form of pyrimidine dimers and 6-4 photoproducts (see Friedberg, E. C., Nature, 421:436-440 (2003)). According to the American Cancer Society, UV radiation induced DNA mutation is one of the leading causes of skin cancer, with more than one million cases diagnosed annually resulting in 11,590 deaths in the United States.

The demand for skin protection agents against the harmful influence of UV solar radiation has become increasingly important in light of the depletion of the ozone layer (van der Leun et al., Influences of ozone depletion on human and animal health, Ed. Tevini M., Ann Arbor: Lewis Publishers, 1993, pp 95-123; and Smith et al., Science, 255:952-959 (1992)). Sunscreens, which work by combining organic and inorganic ingredients to reflect, scatter, or absorb UV radiation, provide significant protection against the damage from solar UV. Early sunscreens developed with inorganic UV filters, such as titanium dioxide (TiO₂) and zinc oxide (ZnO) particles, were often opaque giving the skin a white tinge, which made them unappealing to consumers (Wolf et al., Clin Dermatol., 19:452-459 (2001)). With enhanced UV protection and low opacity, nanosize metal oxide particles have been introduced into cosmetics products in recent years and thousands of tons of nanomaterials are currently applied onto the faces and hands of hundreds of millions of people every year (Nohynek et al., Crit Rev Toxicol 37:251-277 (2007)). With increased popularity, the safety of these metal-based nanoparticles and potential toxicity is under significant debate. Many studies indicated that when applied to skin for less than 8 hours, inorganic nanoparticle filters do not penetrate through the stratum corneum (SC) layer of the skin (Durand et al., Int J Cosmet Sci, 31:279-292 (2009); Gontier et al., Nanotoxicology, 2:218-231 (2009); Newman et al., J Am Acad Dermatol, 61:685-692 (2009); and Oberdorster, E., Environ Health Perspect, 112:1058-1062 (2004)). However, these studies typically examine the effects of nanoparticles greater than 20 nm, and are based on healthy skin samples. Studies evaluating the penetration of ultrafine nanoparticles found that TiO₂, maghemite, and iron nanoparticles less than 15 nm are capable of penetrating through the SC (See Baroli et al., J Invest Dermatol, 127:1701-1712 (2007) and Menzel et al., Nucl Instrum Methods Phys Res Sect B, 219-220: 82-86 (2004)). Other studies have also observed penetration of 4 nm and 60 nm Ti0₂ particles through healthy skin in hairless mice after prolonged exposure from 30-60 days (Wu et al., Toxicol Lett, 191:1-8 (2009)). This penetration leads to increased aging of skin, pathological effects in the liver, and particle accumulation in the brain. Studies like these have raised significant concerns about the prolonged use of these metal oxide nanoparticles for cosmetic applications which lead to investigation of alternative organic filters.

The properties of materials at the nanoscale differ significantly from those at a larger scale, and safety claims by cosmetics manufacturers based on their bulk properties pose great risk without proper federal regulation of their applications (see Buzea et al., Biointerphases, 2:MR17-MR71 (2007)). When decreasing size to the nanoscale, materials alter many of their physical and chemical properties, including but not limited to color, solubility, material strength, electrical conductivity, magnetic behavior, mobility (within the environment and within the human body), chemical and biological activities (Oberdorster et al., Environ Health Perspect, 113:823-839 (2005)). The increased surface to volume ratio also enhances chemical activity, which can result in the increased production of reactive oxygen species (ROS) (Nel et al., Science, 311:622-627 (2006)). ROS production, which has been found in metal oxide nanoparticles, carbon nanotubes, and fullerenes, is the leading force of oxidative stress, inflammation, and consequent damage to DNA, proteins, and membranes (Nel et al., 2006). Further concern for these nanomaterials in applications is their photoactivity when exposed to UV light, which results in greater ROS and free radical production (Dunford et al., FEBS Lett, 418:87-90 (1997)). TiO₂ nanoparticles have been shown to cause far greater damage to DNA than does TiO₂ of larger particle size (Donaldson et al., Toxicol Lett, 88:293-298 (1996)). While 500 nm TiO₂ particles have some ability to cause DNA strand breakage, 20 nm TiO₂ nanoparticles are capable of causing complete destruction of super-coiled DNA, as demonstrated in a plasmid DNA assay, even at lower doses and without exposure to UV. In addition to the increased potential for DNA damage from engineered metal oxide nanoparticles, another concern for their application in cosmetics is the potential for inhalation, ingestion, and penetration through the skin Once in the blood stream, nanomaterials can be circulated inside the body and are taken up by organs and tissues such as the brain, liver, spleen, kidney, heart, bone marrow, and nervous system (Oberdorster et al., 2005). With their stability, the damage of these nanoparticles to human tissues and organs can occur through a traditional ROS pathway, or through accumulation that can impair their normal functions. In vitro studies on BRL 3A rat liver cells exposed to 100-250 μg/ml of Fe₃O₄, Al, Mo0₃ and TiO₂ nanoparticles revealed significant damage from ROS in these cells (Hussain et al., Toxicol In Vitro, 19:975-983 (2005)). Carbon nanotubes have also been shown to be toxic to kidney cells and inhibit cell growth (Oberdorster et al., Part Fibre Toxicol, 2:8 (2005)). The stability of nanomaterials in the environment has also been linked to brain damage and mortality in several aquatic species (Luo, J., Journal of the US SJWP 2:1-16 (2007) and Zhu et al., Mar Environ Res, 62 (Suppl):S5-S9 (2006)).

Due to the potential toxicity associated with prolonged use of metal oxide nanoparticle sunscreens, alternative ingredients that are non-toxic and effective at blocking UV are desired. Ideally, these ingredients should be biodegradable, and less toxic to mammalian cells than metal oxide nanoparticles. The compositions and methods disclosed herein address these and other needs.

### SUMMARY

In accordance with the purposes of the disclosed materials, compounds, compositions, kits, and methods, as embodied and broadly described herein, the disclosed subject matter relates to cosmetic compositions, methods of making said compositions, and methods of using said compositions. In a further aspect, the disclosed subject matter relates to ivy derived nanoparticles that can be used for or in cosmetic compositions. Methods for isolating the ivy derived nanoparticles and preparing cosmetic compositions containing the nanoparticles are also disclosed. Further disclosed are methods of using the compositions described herein for preventing or treating UV radiation damage to the skin of a subject (e.g., as sunscreens).

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### DESCRIPTION OF DRAWINGS

FIG. 1 is a picture depicting the morphologies of ivy nanoparticles observed by AFM.

FIG. 2 contains graphs showing the size distributions of ivy nanoparticles in water, solutions SEC-1 (Panel A) and D220D1 (Panel B), obtained by dynamic light scattering.

FIG. 3 contains UV-Vis extinction spectra of ivy nanoparticles in the samples prepared by size exclusion chromatography (Panel A) and filtration (Panel B). High concentration (low concentration, low dialysis) represents the difference between the extinction spectra found in D220D1 (D220D2, ND220) and D20D1 (D20D2, ND20).

FIG. 4 is a graph displaying the original extinction spectra of the filtered samples.

FIG. 5 is a graph displaying fitting curves considering scattering and absorption extinction.

FIG. 6 contains graphs displaying the comparison of the UV-Vis extinction spectra of TiO₂, ZnO nanoparticles, and ivy nanoparticles. Panel A shows the relative UV-Vis extinction characteristics with the same extinction value at 280 nm. Panel B shows the extinction behaviors at the same concentration (4.92 μg/mL).

FIG. 7 contains graphs of the cytotoxicity analysis using HeLa cells incubated with or without nanoparticles. Panel A shows representative flow cytometry plots for each of the three samples: negative control (Neg control), ivy nanoparticle (Ivy), TiO₂ nanoparticle (TiO₂). Panel B displays the percentage of cells experiencing apoptosis in the three samples. Each point represents an average of 3 samples from one of three experiments. * denotes significant difference based on Student's t test (p<0.05).

FIG. 8 shows pictures demonstrating the biodegradability of ivy nanoparticles as analyzed by AFM using the tapping mode. The isolated ivy nanoparticles were incubated without (Panel A) or with (Panel B) Proteinase K at 37° C. for 30 min.

FIG. 9 contains graphs depicting the distribution of nanoparticles in the subcutaneous (SC) layer of skin Panels A and B show computational simulation results for the distribution of nanoparticles in the SC layer of human skin 8 hours (Panel A) and 20 hours (Panel B) after application to the surface of the skin

### DETAILED DESCRIPTION

The materials, compounds, compositions, articles, and methods described herein may be understood more readily by reference to the following detailed description of specific aspects of the disclosed subject matter and the Examples included therein.

Before the present materials, compounds, compositions, articles, devices, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### Definitions

In this specification and in the claims that follow, reference will be made to a number of terms, which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “the compound” includes mixtures of two or more such compounds, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed, then “less than or equal to” the value, “greater than or equal to the value,” and possible ranges between values are also disclosed, as appropriately understood by the skilled artisan. For example, if the value “10” is disclosed, then “less than or equal to 10” as well as “greater than or equal to 10” is also disclosed. It is also understood that throughout the application data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human.

By “reduce” or other forms of the word, such as “reducing” or “reduction,” is meant lowering of an event or characteristic (e.g., cell toxicity). It is understood that this is typically in relation to some standard or expected value, in other words it is relative, but that it is not always necessary for the standard or relative value to be referred to. For example, “reduces cell toxicity” means lowering the amount of toxicity of cells relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

By “treat” or other forms of the word, such as “treated” or “treatment,” is meant to administer a composition or to perform a method in order to reduce, prevent, inhibit, or eliminate a particular characteristic or event (e.g., damage caused by UV radiation). The term “control” is used synonymously with the term “treat.”

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

Reference will now be made in detail to specific aspects of the disclosed materials, compounds, compositions, articles, and methods, examples of which are illustrated in the accompanying Examples.

### Ivy Derived Nanoparticles

In one aspect, disclosed herein are nanoparticles derived from natural sources, specifically, ivy plants belonging to the genus Hedera. Examples of suitable ivy plants include English ivy (H. helix), Boston ivy (Parthenocissus, Ampelopsis, and tricuspidata), and plants referred to as “poison” ivy. The nanoparticles are secreted from the ivy rootlets. These nanoparticles can be extracted from the ivy rootlets using, for example, solvent extraction, and isolated from other extracted components by using size exclusion chromatography (SEC), high performance liquid chromatography (HPLC), or a combination of these methods.

The nanoparticles described herein have an average particle diameter of less than about 1 micron (1μ, such as less than about 800 nanometers (800 nm), less than about 500 nanometers (500 nm), and less than about 100 nanometers (100 nm). In particular embodiments, the nanoparticles can have an average particle diameter generally ranging from about 1 nm to less than about 1000 nm, such as from about 10 nm to about 500 nm, about 15 nm to about 200 nm, about 20 nm to about 100 nm, about 30 nm to about 90 nm, and about 50 nm to about 80 nm. For example, the nanoparticles described herein can have an average particle diameter of about 10 nm, about 20 nm, about 30 nm, about 40 nm, about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, or about 200 nm, where any of the stated values can form an upper or lower endpoint when appropriate. In further examples, the nanoparticles, which are typically uniform in shape, can have an average particle diameter of approximately 70 nm. The size of the nanoparticles can be measured according to methods as known in the art, including, for example, atomic force microscopy (AFM), scanning electron microscopy (SEM), transmission electron microscopy (TEM), x-ray diffraction (XRD), and dynamic light scattering (DLS). Preferably, AFM and/or DLS are used to determine the size of the nanoparticles. DLS provides the hydrodynamic sizes of the particles. In general, the hydrodynamic sizes are usually greater than the sizes of dry particles obtained by AFM due to the polydispersity of the nanoparticles (see Hoo et al., J. Nanopart. Res. 10:89 (2008) and Zanetti-Ramos et al., Mater. Sci. Eng. C-Biomimetic. Supramol. Syst. 29:638 (2009)). Large particles scatter a significant amount of light so that the diameters given by DLS intensity distributions can be greater than that obtained by AFM even if there are only a few large particles.

The nanoparticles obtained from natural sources can be isolated as mixtures of compounds. In some embodiments, the nanoparticles contain one or more organic compounds. The organic compounds typically include carbon, hydrogen, and nitrogen atoms, and can further include oxygen, chlorine, and/or sulfur atoms. Exemplary empirical formulas describing the chemical compositions of the organic compounds include, for example, one or more of C₂₆H₄₂N₈O₁₀, C₁₈H₈ClNO₁₁S₅, C₂₁H₄₆N₂₀O₃, C₃₈H₇₆N₂O₃, C₁₄H₆Cl₃NO₁₂S₃, C₂₀H₇N₅O₁₁S₅, C₃₈H₇₄N₂O₄, C₂₈H₄₆N₆, C₃₈H₇₆N₂O₅, C₃₈H₇₆N₂O₃, C₂₃H₂₆N₁₂O₁₀, C₃₂H₃₃N₇OS₃, C₂₉H₂₈N₁₂O₁₀, C₃₈H₇₆N₂O₃, C₃₇H₆₃N₅O₃, C₂₁H₂₄N₁₂O₅S, C₃₈H₇₆N₂O₂, C₂₁H₄₅N, and C₁₈H₂₃N₅O₂S₂, as determined, for example, by analytical chromatographic and mass spectroscopic methods (e.g., HPLC/MS) as described in Zhang et al., Nano Lett., 8 (5):1277-1280 (2008). In some examples, the nanoparticles include one or more of these chemical compositions. For example, the nanoparticles can include 19 organic compounds of differing chemical compositions in amounts that combine to equal 100% by weight of the organic compounds present.

The nanoparticles can be used as isolated (e.g., as a mixture of organic compounds) or can be purified to result in nanoparticles comprising predominantly one organic compound. For example, the nanoparticles can include at least 60% by weight of an organic compound of the structure C₂₆H₄₂N₈O₁₀ and the remaining 40% by weight can include one or more organic compounds of a different structure. In some examples, the nanoparticles can include at least 65% of one organic compound, at least 70% of one organic compound, at least 75% of one organic compound, at least 80% of one organic compound, at least 85% of one organic compound, at least 90% of one organic compound, at least 95% of one organic compound, or at least 99% of one organic compound. In some examples, the nanoparticles can be isolated in the following matter. Shoots from the genus Hedera can be sterilized using bleaching agents, detergents, or a combination of these. An advantage of this technique is that the aerial rootlets grown in this culture system can be sterile and free from any environmental contaminants. Optionally, the shoots can be de-leaved and trimmed prior to sterilization. The sterilized shoots can then be grown under irradiance for a period of time in order to reach about 3 cm in length. In some examples, the shoots can be grown for about 12 hours or more, about 18 hours or more, about 24 hours or more, about 36 hours or more, about 48 hours or more, or about 72 hours or more. After the shoots reach about 3 cm in length, the aerial roots can be transferred to a container holding medium (e.g., MS medium) and optionally stored for a period of time (e.g., about 1 day, about 2 days, about 3 days, about 4 days, about 5 days, about 6 days, about 7 days, about 8 days, about 9 days, about 10 days, about 11 days, about 12 days, about 13 days, about 14 days, or more than about 14 days). Following this, the tips of the rootlets can then be homogenized in water and centrifuged or filtered to remove residual matter. Optionally, the supernatant or filtrate can be dialyzed to remove compounds with a molecular weight less than about 13,000 Daltons (e.g., less than about 12,500, less than about 12,000, less than about 11,000, less than about 10,000, less than about 9,000, less than about 8,000, less than about 7,000, less than about 6,000, or less than about 5,000 Daltons) to isolate the ivy nanoparticles.

The nanoparticles can be purified based on differences in solubility between the nanoparticles and other materials using extraction (e.g., liquid-liquid extraction. In some examples, the nanoparticles can be filtered by size and/or density, using, for example, flow cytometry, density gradient centrifiguation, filtration, and diafiltration methods. In some examples, the nanoparticles can be filtered through one or more filters or membranes. For example, the nanoparticle-containing solution can be filtered through a 100-300 nm filter (e.g., a 220 nm filter), a 5-99 nm filter (e.g., a 20 filter), or a combination of these. In some examples, the nanoparticle-containing solution is successively filtered through filters of varying sizes. Additional methods of purifying the nanoparticles include chromatographic methods such as high performance liquid chromatography (HPLC), size exclusion chromatography, or combinations of these. In these methods, the purification of the nanoparticles can be monitored by the elution times and the shape of the UV peak at 280 nm. Any of the above listed methods of purifying the nanoparticles can be used separately or in combination (e.g., successively). A preferred method of purifying the nanoparticles includes using a combination of filtration, diafiltration, and size-exclusion chromatography. Further examples of methods to isolate and purify the nanoparticles are described, for example, in Li et al., Nanoscale Res Lett, 5:1487-1491 (2010); Xia et al., Journal of Nanobiotechnology, 8:12 (2010); and Zhang et al., Nano Letters, 8 (5):1277-1280 (2008), which are hereby incorporated by reference in their entireties.

### Cosmetic Compositions

Cosmetic compositions and methods for preparing and using these compositions are provided herein. These compositions are useful in protecting of the skin of a subject against damage caused by exposure to thermal, biological, chemical, occupational, and environmental factors. Specifically, compositions including nanoparticles derived from ivy are provided.

The compositions described herein also include one or more cosmetically acceptable materials. These materials include, for example, anti-UV agents, anti-foaming agents, antiperspirants, bactericides, chelating agents, colorants, complexing agents, conditioning agents, deodorants, diluents, dispersants, dyes, emollients, emulsifiers, fats, fillers, flavorants, humectants, insect repellants, moisturizers, odorants, oils, perfumes, pH adjusters, pigments, plasticizers, preservatives, skin protectants, softeners, solvents, stabilizers, sunscreens, thickening agents, waxes, and mixtures thereof. The compositions can also include complexing agents, alcohols, polyols, polymers, foam stabilizers, electrolytes, organic solvents or silicone derivatives, or plant extracts.

In some embodiments, the compositions contain ultraviolet (UV) radiation absorbing substances, including UVA and/or UVB filters. The UVA and UVB filters can be soluble in water or oil. Examples of oil-soluble UVA filters include triazines and triazoles. An example of a water-soluble UVA filter for used with the compositions described herein is 2′-bis(1,4-phenylene)-1H-benzimidazole-4,6-disulfonic acid sodium salt.

Oil-soluble UVB filters that can be used herein include 3-benzylidenecamphor derivatives, 4-aminobenzoic acid derivatives, cinnamic acid esters, salicylic acid esters, benzophenone derivatives, benzalmalonic acid esters, 2-cyano-3,3-diphenylacrylic acid esters, diethylhexyl-butamidotriazone, and 2,4,6-trianilino-(p-carbo-2′-ethyl-1′-hexyloxy)-1,3,5-triazine. Water-soluble UVB filters include, for example, 2-phenylbenzimidazole-5-sulfonic acid and salts thereof, sulfonic acid derivatives of benzophenones, and sulfonic acid derivatives of 3-benzylidene camphor.

The compositions described herein can further include organic sunscreen agents having at least one chromophoric group absorbing within the ultraviolet ranging from 290 to 400 nm. Chromophoric organic sunscreen agents can include p-aminobenzoic acid, its salts and its derivatives (ethyl, isobutyl, glyceryl esters; p-dimethylaminobenzoic acid); anthranilates (o-aminobenzoates; methyl, menthyl, phenyl, benzyl, phenylethyl, linalyl, terpinyl, and cyclohexenyl esters); salicylates (octyl, amyl, phenyl, benzyl, menthyl, glyceryl, and dipropyleneglycol esters); cinnamic acid derivatives (menthyl and benzyl esters, α-phenyl cinnamonitrile; butyl cinnamoyl pyruvate); dihydroxycinnamic acid derivatives (umbelliferone, methylumbelliferone, methylaceto-umbelliferone); trihydroxycinnamic acid derivatives (esculetin, methylesculetin, daphnetin, and the glucosides, esculin and daphnin); hydrocarbons (diphenylbutadiene, stilbene); dibenzalacetone and benzalacetophenone; naphtholsulfonates (sodium salts of 2-naphthol-3,6-disulfonic and of 2-naphthol-6,8-disulfonic acids); dihydroxy-naphthoic acid and its salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyquinoline salts, 2-phenylquinoline); hydroxy- or methoxy-substituted benzophenones; uric and vilouric acids; tannic acid and its derivatives (e.g., hexaethylether); (butyl carbityl) (6-propyl piperonyl) ether; hydroquinone; benzophenones (oxybenzone, sulisobenzone, dioxybenzone, benzoresorcinol, 2,2′,4,4′-tetrahydroxybenzophenone, 2,2′-Dihydroxy-4,4′-dimethoxybenzophenone, octabenzone; 4-isopropyldibenzoylmethane; butylmethoxydibenzoylmethane; etocrylene; and 4-isopropyl-dibenzoylmethane).

Particularly useful organic sunscreen agents are: 2-ethylhexyl p-methoxycinnamate, 4,4′-t-butyl methoxydibenzoylmethane, 2-hydroxy-4-methoxybenzophenone, octyldimethyl p-aminobenzoic acid, digalloyltrioleate, 2,2-dihydroxy-4-methoxybenzophenone, ethyl 4-[bis(hydroxypropyl)]aminobenzoate, 2-ethylhexyl-2-cyano-3,3-diphenylacrylate, 2-ethylhexylsalicylate, glyceryl p-aminobenzoate, 3,3,5-trimethylcyclohexylsalicylate, methylanthranilate, p-dimethylaminobenzoic acid or aminobenzoate, 2-ethylhexyl p-dimethylaminobenzoate, 2-phenylbenzimidazole-5-sulfonic acid, 2-(p-dimethylaminophenyl)-5-sulfoniobenzoxazoic acid and mixtures thereof. Amounts of the organic sunscreen agent can range from about 0.1 to about 5%, for example from about 0.2 to about 2%, from about 0.6 to about 1.5%, or from about 0.8 to about 1.25% by weight.

Active substances can also be included in the compositions described herein. These active substances include anti-acne agents, anti-inflammatory agents, anti-irritants, antioxidants, radical scavengers, and mixtures thereof suitable for use in cosmetics. Exemplary active substances include vitamins A, B3, B5, B6, B8, C, E, or PP, niacin, carotenoids (e.g., β-carotene, lycopene, astaxanthin, zeaxanthin, lutein, and flavonoids such as catechins, hesperidin, proanthocyanidins and anthocyanins), polyphenols, and minerals (e.g., zinc, calcium, and magnesium). One or more prebiotics can also be included in the compositions described herein. Suitable prebiotics include, for example, oligosaccharides produced from glucose, galactose, xylose, maltose, sucrose, lactose, starch, xylan, hemicellulose, inulin, gums of acacia type, or mixtures of these. Active substances for use in the compositions described herein further include anti-cellulite agents, tanning agents, skin lightening agents, skin soothing agents, skin healing agents, antimicrobial agents, and antifungal agents.

Active substances for use in the compositions described herein also include hydrophilic and lipophilic substances. Hydrophilic active substances of proteins or protein hydrolysates, amino acids, polyols (e.g., glycerol, sorbitol, butylene glycol and polyethylene glycol), urea and derivatives (e.g., hydroxyalkylurea derivatives), allantoin, sugars and sugar derivatives, starch, or bacterial or plant extracts (e.g., aloe vera extracts) can also be included in the compositions described herein. Suitable lipophilic active substances include some of the active substances mentioned above including retinol (vitamin A) and derivatives and tocopherol (vitamin E) and derivatives, and also includes ceramides, essential oils, and nonsaponifiable materials (e.g., tocotrienol, sesamin, γ-oryzanol, phytosterols such as stigmasterol, β-sitosterol, and campesterol, squalenes, waxes, or terpenes).

Moisturizing active substances can also be used in the compositions described herein. Examples include sphingoid-based compounds, glycosphingolipids, phospholipids, essential fatty acids, 1-2-diacylglycerol, 4-chromanone, pentacyclic triterpenes, petrolatum, hyaluronic acid and derivatives, pentanediol, pidolates, serine, xylitol, lactic acid, sodium lactate, glyceryl polyacrylate, ectoine and derivatives, chitosan, oligo- and polysaccharides, cyclic carbonates, N-lauroylpyrrolidonecarboxylic acid, N-α-benzoyl-L-arginine, and steroidal derivatives (e.g., DHEA).

The compositions described herein can be emulsions of a liquid or a semi-liquid. The emulsions can be obtained by dispersing an oil phase into an aqueous phase to form an oil in water (O/W) emulsion. Alternatively, the emulsions can be obtained by dispersing an aqueous phase into an oil phase to form a water in oil (W/O) emulsion. Other suitable emulsion types include, for example, cream emulsions and microemulsions. The oils, emulsifiers, and coemulsifiers for use in the emulsified compositions can be chosen by those of skill in the art.

Suitable emulsifiers for use in the compositions described herein include polyglyceryl-2 dipolyhydroxystearate, PEG-30 dipolyhydroxystearate, cetyldimethicone copolyol, glycol distearate, glycol dilaurate, diethylene glycol dilaurate, sorbitan trioleate, glycol oleate, glyceryl dilaurate, sorbitan tristearate, propylene glycol stearate, propylene glycol laurate, propylene glycol distearate, sucrose distearate, PEG-3 castor oil, pentaerythrityl monostearate, pentaerythrityl sesquioleate, glyceryl oleate, glyceryl stearate, glyceryl diisostearate, pentaerythrityl monooleate, sorbitan sesquioleate, isostearyl diglyceryl succinate, glyceryl caprate, palm glycerides, cholesterol and derivatives, lanolin, glyceryl oleate (containing 40% monoester), polyglyceryl-2 sesquiisostearate, polyglyceryl-2 sesquioleate, PEG-20 sorbitan beeswax, sorbitan oleate, sorbitan isostearate, trioleyl phosphate, glyceryl stearate and ceteareth-20, sorbitan stearate, PEG-7 hydrogenated castor oil, PEG-5 soya sterol, PEG-6 sorbitan beeswax, glyceryl stearate SE, methylglucose sesquistearate, PEG-10 hydrogenated castor oil, sorbitan palmitate, PEG-22/dodecyl glycol copolymer, polyglyceryl-2 PEG-4 stearate, sorbitan laurate, PEG-4 laurate, polysorbate 61, polysorbate 81, polysorbate 65, polysorbate 80, triceteareth-4 phosphate, triceteareth-4 phosphate and sodium C₁₄₋₁₇-alkyl sec sulfonate, glyceryl stearate and PEG-100 stearate, polysorbate 85, trilaureth-4 phosphate, PEG-35 castor oil, sucrose stearate, trioleth-8 phosphate, C₁₂₋₁₅-Pareth-12, PEG-40 hydrogenated castor oil, PEG-16 soya sterol, polysorbate 80, polysorbate 20, polyglyceryl-3 methylglucose distearate, PEG-40 castor oil, sodium cetearyl sulphate, lecithin, laureth-4 phosphate, propylene glycol stearate SE, PEG-25 hydrogenated castor oil, PEG-54 hydrogenated castor oil, glyceryl stearate SE, PEG-6 caprylic/capric glycerides, glyceryl oleate and propylene glycol, glyceryl lanolate, polysorbate 60, glyceryl myristate, glyceryl isostearate and polyglyceryl-3 oleate, glyceryl laurate, PEG-40 sorbitan peroleate, laureth-4, glycerol monostearate, isostearyl glyceryl ether, cetearyl alcohol and sodium cetearyl sulphate, PEG-22 dodecylglycol copolymer, polyglyceryl-2 PEG-4 stearate, pentaerythrityl isostearate, polyglyceryl-3 diisostearate, sorbitan oleate and hydrogenated castor oil and cera alba and stearic acid, sodium dihydroxycetyl phosphate and isopropyl hydroxycetyl ether, methylglucose sesquistearate, methylglucose dioleate, sorbitan oleate and PEG-2 hydrogenated castor oil and ozokerite and hydrogenated castor oil, PEG-2 hydrogenated castor oil, PEG-45/dodecylglycol copolymer, methoxy PEG-22/dodecylglycol copolymer, hydrogenated cocoglycerides, polyglyceryl-4 isostearate, PEG-40 sorbitan peroleate, PEG-40 sorbitan perisostearate, PEG-8 beeswax, laurylmethicone copolyol, polyglyceryl-2 laurate, stearamidopropyl PG dimonium chloride phosphate, PEG-7 hydrogenated castor oil, triethyl citrate, glyceryl stearate citrate, cetyl phosphate polyglycerol methylglucose distearate, poloxamer 101, potassium cetyl phosphate, glyceryl isostearate, and polyglyceryl-3 diisostearates. The coemulsifiers for use in the emulsified compositions described herein include butyloctanol, butyldecanol, hexyloctanol, hexyldecanol, octyldodecanol, behenyl alcohol, cetearyl alcohol, and lanolin alcohols.

The oil phase of the emulsions can include esters of saturated and/or unsaturated, branched and/or unbranched alkanecarboxylic acids with a chain length from 3 to 30 carbon atoms; saturated and/or unsaturated, branched or unbranched alcohols with a chain length from 3 to 30 carbon atoms, from the group of the esters of aromatic carboxylic acids; and saturated and/or unsaturated, branched and/or unbranched alcohols with a chain length from 3 to 30 carbon atoms. Exemplary ester oils include isopropyl myristate, isopropyl palmitate, isopropyl stearate, isopropyl oleate, n-butyl stearate, n-hexyl laureate, n-decyl oleate, isooctyl stearate, isononyl stearate, isononyl isononanoate, 2-ethylhexyl palmitate, 2-ethylhexyl laureate, 2-hexyldecyl stearate, 2-octyldocecyl palmitate, oleyl oleate, oleyl erucate, erucyl oleate, erucyl erucate, as well as synthetic, semisynthetic, and natural mixtures of such esters, for example, jojoba oil. Additional oil phase components include branched and unbranched hydrocarbons and hydrocarbon waxes, silicone oils, dialkylethers, the group of saturated or unsaturated, branched or unbranched alcohols, as well as the fatty acid triglycerides, namely the triglycerine ester of saturated and/or unsaturated, branched and/or unbranched alkanoic acids. The fatty acid triglycerides can include, for example, synthetic, semisynthetic, and natural oils such as olive oil, sunflower oil, soy bean oil, peanut oil, rape seed oil, almond oil, palm oil, coconut oil, and palm kernel oil.

The aqueous phase of the emulsified compositions can include alcohols (e.g., ethanol or isopropanol), diols (e.g., 1,2-propanediol or 2-methyl-1,3-propanediol), or polyols having a low number of carbon atoms, and also ethers thereof, including ethanol, isopropanol, propylene glycol, glycerin, ethyleneglycol, ethyleneglycol monoethyl- or -monobutyl ether, propylene glycolmonomethyl, -monoethyl-, or -monobutyl ether, diethyleneglycol monomethyl- or -monoethyl ether.

The compositions described herein can be provided in all the formulation forms normally available for the method of administration selected. As such, the one or more cosmetically acceptable materials can be of various natures depending on the type of composition considered. In some examples, the composition is prepared as a formulation for topical administration. The compositions for topical administration described herein can be aqueous solutions, aqueous/alcoholic solutions, oily solutions, dispersions, emulsions, aqueous or anhydrous gels, microcapsules, microparticles, or ionic or non-ionic vesicular dispersions. For example, the compositions can be formulated as an ointment, a liquid, a paste, a cream, a lotion, a foam, a gel, an emulsion, a powder, a shampoo, a conditioner, a hair rinse, a hair tonic, a hair spray, or a hair care treatment.

The compositions described herein can include protecting sun creams for the face, for the hands, for the feet, for the major anatomical folds, or for the body. The compositions described herein can further include cleansing, treating, or care creams for the face, for the hands, for the feet, for the major anatomical folds or for the body (e.g., day creams, night creams, make-up-removing creams, or cream foundations), make-up products, such as liquid foundations, make-up-removing milks, protective or care body milks, aftersun milks, lotions, gels or foams for caring for the skin, such as cleansing or disinfecting lotions, sun lotions, artificial tanning lotions, bath compositions, deodorant compositions comprising a bactericidal agent, aftershave gels or lotions, depilatory creams, or compositions for combating insect stings and bites. Formulations for the compositions described herein can optionally include solid formulations such as cleansing soaps and bars. These compositions can also be formulated for scalp administration in the form of solutions, creams, gels, emulsions, foams, or aerosols. The compositions can further be formulated as a component of a polymer matrix, a skin covering, a wound covering, a bandage, a wipe pad, or a spray.

### Methods of Making the Cosmetic Compositions

A method of preparing a composition is also described herein. The method includes mixing the ivy derived nanoparticles with one or more cosmetically acceptable materials. The method of preparing the composition can optionally include the steps of isolating the nanoparticles from the ivy and purifying the nanoparticles according to the methods described herein prior to mixing the nanoparticles with one or more cosmetically acceptable materials. As described above, the nanoparticles can be isolated from the ivy using extraction techniques as known in the art for extracting natural materials from plants. Further, the method can optionally include the steps of treating the composition with an emulsifier to provide an emulsified composition. The method can also include the step of formulating the composition for topical administration.

### Methods of Delivery

Also described herein are methods of delivering the compositions containing the nanoparticles described herein and one or more cosmetically acceptable materials. The method includes applying the composition to the skin of a subject. The composition can be applied to any part of the external portion of the external portion of the skin and/or nails. For example, the composition can be applied to the face, lips, under-eye area, eyelids, scalp, neck, torso, arms, hands, legs, or feet. The application can be done using, for example, the palms or fingers of the hands or an implement (e.g., a cotton ball, a swab, or a pad). Further, the application can be performed using aerosol sprays.

### Methods of Use

Further described herein are methods of protecting the skin of a subject from damage resulting from UV exposure (e.g., from sunlight). The methods include applying the composition described herein to the skin of a subject. Exemplary subjects for use with the methods described herein include those exposed to sunlight (e.g., excessive sunlight) or those having a disease or condition that causes skin sensitivity to ultraviolet light or radiation (e.g., albinism, systemic lupus erythematosus, porphyrias, vitiligo, or xeroderma pigmentosa). In some examples, the skin is protected from sunlight with an average SPF of at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more than 50. In these methods, the nanoparticles often do not penetrate into the skin, thus resulting in lower cell toxicity than metal based nanoparticles (e.g., TiO₂ nanoparticles). In some embodiments, the nanoparticles do not penetrate more than about 10 μm, about 20 μm, about 25 μm, about 30 μm, about 35 μm, about 40 μm, about 45 μm, or about 50 μm into the skin with one application. Further, the nanoparticles described herein are biodegradable, thus posing fewer environmental risks than compositions including metal based nanoparticles. In these methods, the composition is administered topically as an ointment, a paste, a cream, a lotion, a foam, a gel, an emulsion, or a powder.

Administration of the compositions described herein can be carried out using cosmetically effective amounts of the compositions for periods of time effective to treat or protect the intended area. The effective amount of the compositions can be determined by one of ordinary skill in the art and includes exemplary quantities for a subject of from about 0.1 mg/cm² to about 20 mg/cm² skin The cosmetic treatment and prevention methods described herein can include a single administration or multiple administrations. In some examples, the administration is repeated, for example 2 or 3 times daily over one or more days. This administration can be over an extended period during the subject's lifetime with one or more periods of interruption as needed. For example, administration can occur for a period of at least 1 week, at least 1 month, at least 3 months, at least 6 months, at least 1 year, at least 5 years, at least 10 years, or at least 20 years. Those of skill in the art will understand that the specific quantity and frequency of administration can be varied and will depend upon a variety of factors, including planned exposure, the activity of the specific composition employed, formulation for administration, and severity of the condition.

### Kits

Also provided herein are kits for the cosmetic treatment of skin or to produce a desired cosmetic result (e.g., UV protection). A kit can include a plurality of ivy nanoparticles as described herein and can further include one or more cosmetically acceptable materials. For example, a kit can include a plurality of English ivy derived nanoparticles and a carrier. A kit can further include one or more UV protective agents. A kit can include a topical formulation of the compositions as described herein. A kit can additionally include directions for use of the kit (e.g., instructions for application to a subject).

The examples below are intended to further illustrate certain aspects of the methods and compounds described herein, and are not intended to limit the scope of the claims.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1

### Ivy Nanoparticle Isolation

Fresh-grown rootlets of English ivy were collected before attaching themselves to solid surfaces. The collected rootlets were meshed using tweezers in 5 mL clean tube containing 500 μL of 20 nm filtered water after washing them three times with ddH₂O. The solution was then centrifuged at 3000 RPM for 5 min, and the supernatant was collected. Two-thirds of the solution was dialyzed overnight at 4 degrees with ddH₂O through a cellulose membrane (Sigma Aldrich, D9277; St. Louis, Mo.) allowing free pass of chemicals less than 12,500 Daltons. Size exclusion chromatography and high-performance liquid chromatography (SEC-HPLC) were used to isolate the nanoparticles from the solution to separate the nanoparticles in solution based on their sizes (Wei et al., Anal. Chem., 71:2085 (1999)). The Varian HPLC system consists of three components including Varian Prostar 210, the Varian Prostar 335 detector, and the Varian Prostar 430 autosampler. Next, 200 μL of dialysis solution was loaded onto the column (PHENOMENEX™ BIOSEP-SECS3000 column with an attached Security Guard™ cartridge system). The running speed of the mobile phase was set to 0.5 mL/min, and the back pressure of the column was 580 psi. Samples were collected depending on the retention time and labeled as SEC-1, SEC-2, and SEC-3, with the time sequence. Filtration was another method used to reveal the optical properties of ivy nanoparticles. The dialyzed solution was filtered through the Millipore membrane (220-nm Nylon filters) and was labeled as D220D1. Half of this solution was filtered again through 20-nm filters and was labeled as D20D1. Furthermore, a portion of each solution was diluted into ⅓ concentrations. The diluted solutions were labeled D220D2 and D20D2, respectively. The non-dialysis solutions were also prepared and labeled ND220 and ND20, respectively.

### Example 2

### Optical Absorption and Scattering Properties

The morphologies of the ivy nanoparticles isolated according to the methods described in Example 1 were observed by Agilent 5500 atomic force microscope (Agilent Technologies, Santa Clara, Calif.). The instrument was operated at room temperature (20° C.) using PicoView™ (Agilent Technologies, Santa Clara, Calif.) in AC mode, which means little or no contact between the tip and the sample. Dynamic light scattering (DLS) measurements were performed by a Brookhaven Instruments BI-200SM goniometer equipped with a PCI BI-9000AT digital correlator (Brookhaven Instruments Corporation; Holtsville, N.Y.). The operating wavelength was 633 nm and the detector was located at the scattering angle of 90°. The ultraviolet and visible (UV-Vis) extinction (absorption and scattering) spectra were measured by the Thermo Scientific Evolution 600 UV-Visible spectrophotometers (Thermo Fisher Scientific; Waltham, Mass.), using a quartz cuvette with an optical length of 10 mm.

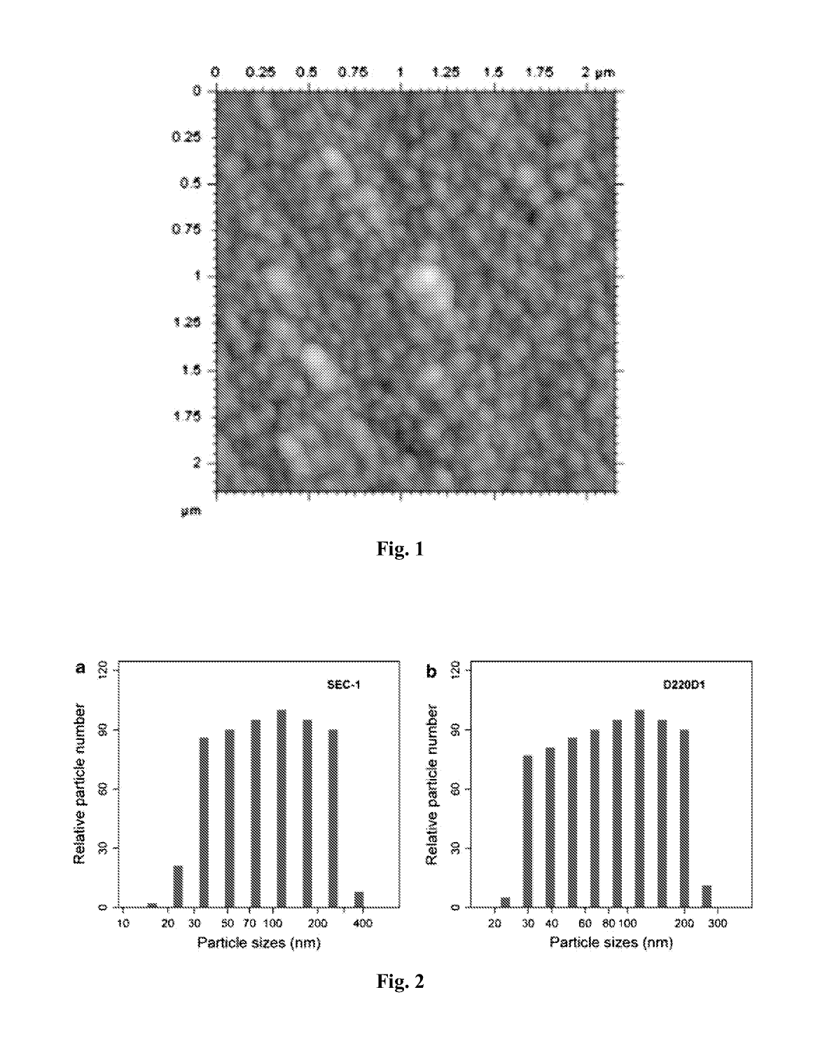

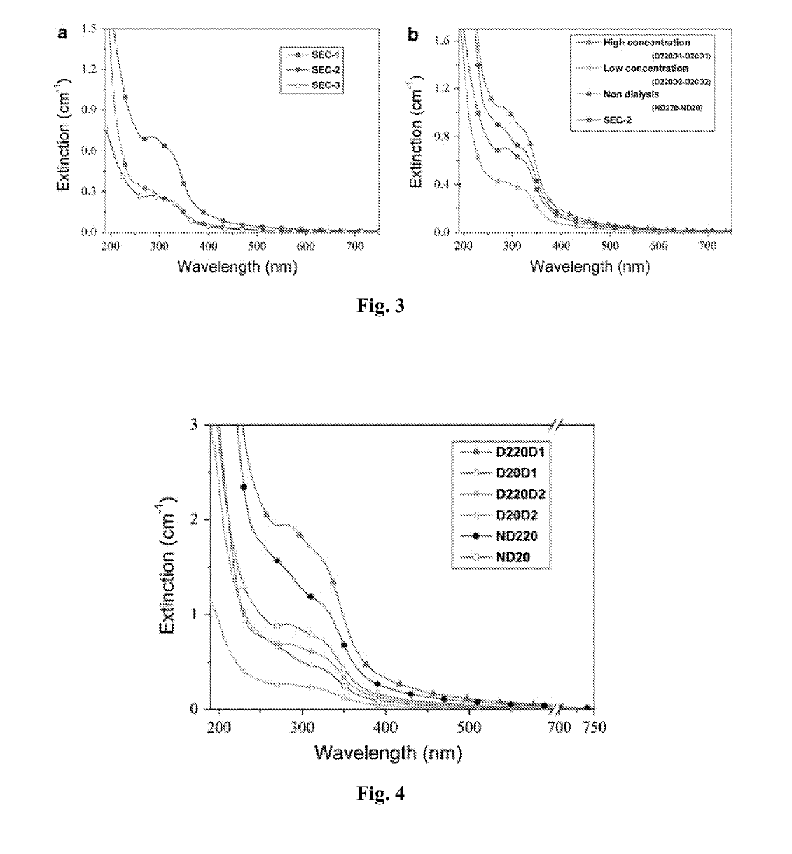

Dry ivy nanoparticles were first imaged using atomic force microscopy (AFM), as shown in FIG. 1. The average diameter of the ivy nanoparticles was 68.3 nm, similar to that of the measurements reported in Zhang et al., Nano Lett., 8:1277 (2008). The sizes of nanoparticles were not uniform in AFM image. Furthermore, size distributions of the ivy nanoparticles were observed in the samples of SEC-1, SEC-2, D220D1, and ND220 by dynamic light scattering (DLS) measurement. No size distribution was found in the sample of SEC-3. The nanoparticles in solutions were the polydisperse system. The sizes of nanoparticles ranged from tens of nanometers to several hundred nanoparticles. The average diameters of particles were 117.4 nm for SEC-1, 131.6 nm for SEC-2, 97.8 nm for D220D1, and 112.3 nm for ND220 (see FIG. 2). The extinction spectra of samples from the SEC technology and the filtration are shown in FIGS. 3a and b, respectively. The extinction spectra of ivy nanoparticles using the filtration method were obtained by subtracting the extinction spectra of samples filtered using 20 nm filters from those filtered by 220 nm filters. For example, the curve in FIG. 3b with the symbol of high concentration denotes the difference between the extinction spectra found in D220D1 and D20D1. The spectrum characteristics of those samples from the SEC technology and the filtration were approximately consistent. These phenomena including the DLS results mean that the SEC and the filtration are the effective direct and indirect methods to investigate the optical properties of ivy nanoparticles. These nanoparticles display strong ultraviolet and weak visible light extinction and, interestingly, a sharp transition edge from the ultraviolet to the visible range. A significant extinction band and a shoulder can be found around the wavelengths of 280 nm and 325 nm for all the samples, respectively.

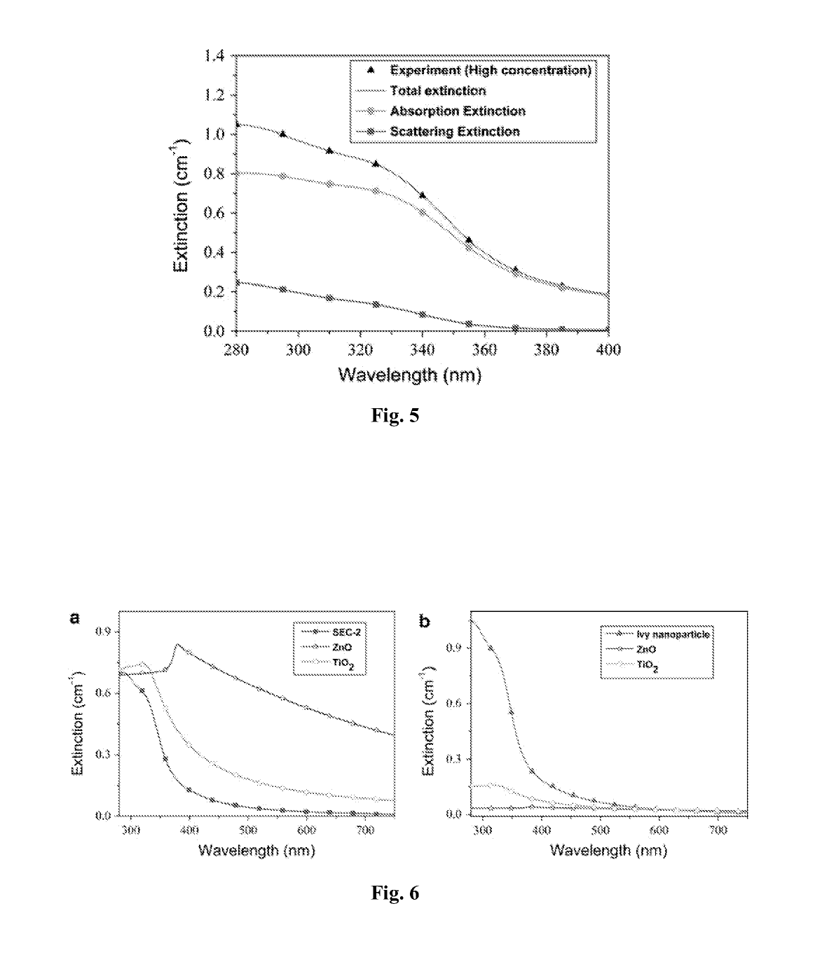

To illustrate the content of the solution, the original spectra are shown before subtraction in FIG. 4. The extinction band and the shoulder were at the same positions not only for the samples filtered through the 220 nm filters but also the ones filtered through the 20 nm filters. This similarity implies that the components in the solutions are similar to that of the ivy nanoparticles. In addition, the nondialysis samples show differences when compared to the dialysis samples in the original absorption spectra in FIG. 5. This can be due to the dialysis process in which some protein or other molecules less than 12,500 Daltons are filtered out. The ingredients of ivy nanoparticles have been analyzed by this research team. Proteins have been confirmed as the dominant compounds of the ivy nanoparticles (Lenaghan et al., manuscript in preparation). The absorption peak at the wavelength of 280 nm is the characteristic for many proteins (Coulter et al., J. Gen. Physiol., 19:739 (1936)). In general, three of the amino acid side chains (Trp, Tyr and Phe) contribute to the UV absorption of a protein at 280 nm. Furthermore, the concentration of ivy nanoparticles in the sample of high concentration (D220D1-D20D1) was measured to be 4.92 μg/ml by the Bradford protein assay (Jahanshahi et al., IEE Proc.-Nanobiotechnol. 151:176 (2004)). Scattering (just as the Tyndall effects shown in DLS experiments) also makes considerable contribution to the total extinction besides the absorption. Herein, extinction behaviors of the ivy nanoparticles were simulated by the Mie scattering theory with the help of the software MieTab 8.38 (Popov et al., J. Phys. D-Appl. Phys., 38:2564 (2005)). While the refractive index of most organic compounds is between 1.3 and 1.73, the refractive index of ivy nanoparticle was set to 1.5. The refractive index of water from 280 nm to 400 nm was from the reported data (Daimon et al., Appl. Opt., 46:3811 (2007)). The extinction coefficients were adjusted to fit the experimental results. The fitting curve of the total extinction, scattering and absorption curves are shown in FIG. 5. From these results, the scattering effect was found to contribute to the extinction, although this contribution was much smaller than that of the absorption. The absorption and scattering make ivy nanoparticles display strong ultraviolet extinction behavior. This behavior offers some potential applications such as sunscreen. The characteristics of extinction spectra of ivy nanoparticles offer advantages in the sunscreen application for blocking the ultraviolet light and maintaining the high visible transparence. Ivy nanoparticles can use their optical absorption and scattering properties to eliminate the ultraviolet light, just as inorganic nanoparticles such as ZnO and TiO₂ do in sunscreen (Dransfield, G. P., Radiat. Prot. Dosim., 91:271 (2000)).

In order to illustrate the relative ultraviolet-visible extinction characteristics, solutions of ZnO nanoparticles (NanoAmor, 99.9%, 90-200 nm) and TiO₂ nanoparticles (NanoAmor, Rutile, 99%, 50 nm) were adjusted to be the same extinction value as SEC-2 at 280 nm. This wavelength is the beginning of UV-B (280-320 nm). Sunscreens protect from both UV-B and UV-A (320-400 nm) irradiation. As shown in FIG. 6a, the ivy nanoparticles displayed a sharper edge from the ultraviolet range to the visible range. Herein, the concentrations of TiO₂ and ZnO suspensions were 23 μg/ml and 100 μg/ml, respectively. After this, the extinction spectra of the above TiO₂ and ZnO suspensions were roughly converted to the spectra with the concentration of 4.92 μg/ml, as shown in FIG. 6b. The ivy nanoparticles (high concentration) exhibited a stronger ultraviolet absorption and sharper decrease near the visible range. Due to the non-toxicity and strong extinction property, ivy nanoparticles are a promising material for sunscreen application.

### Example 3

### Cytotoxicity Study

Although nanoparticles greater than 20 nm in diameter have not been reported to permeate through human skin, this data was obtained using healthy individuals in an optimal setting (Baroli et al., 2007). In specific cases, the skin structure can be changed to allow the penetration of large particles into the blood system, which has been demonstrated by the ability of 1,000 nm particles to access the dermis when intact skin is flexed (Tinkle et al., Environ Health Perspect, 111:1202-1208 (2003)). More frequently, however, when skin is damaged, as in the case of people with sunburn, blemished skin, frequent shaving, or massages, there is an increased risk of penetration (see, for example, Larese et al., Toxicology, 255:33-37 (2009); Mortensen et al., Nano Lett, 8:2779-2787 (2008); and Zhang et al., Toxicol Appl Pharmacol, 228:200-211 (2008)). A recent report, by the US-based Environmental Working Group, on the health risks of commercially available cosmetics and personal care products found that more than half of all cosmetics contained ingredients that act as “penetration enhancers” (see Hoet et al., J Nanobiotechnology, 2:12 (2004)). This raises further concerns for the safety of applied nanoparticles for personal care and cosmetics, since these agents will presumably increase the penetration potential of nanoparticles. As such, the cytotoxicity of nanoparticles should be thoroughly tested before their application in sunscreens.

Due to the increased toxicity associated with internalized nanoparticles, as mentioned earlier, the toxicity of a mammalian endothelial cell line, HeLa cells, was examined. HeLa cells are commonly used for testing the toxicity and trafficking of nanoparticles (see, for example, Park et al., Int J Pharm, 359:280-287 (2008); Chithrani et al., Nanomedicine, 5:118-127 (2009); and Minchin, R., Nat Nanotechnol, 3:12-13 (2008)).

HeLa cells were cultured in a Dulbecco's Modified Eagle Medium (DMEM) (Mediatech Inc., Manassas, Va.) solution supplemented with 10% heat-inactivated fetal bovine serum in a humidified incubator with an atmosphere of 5% CO₂ in air at 37° C. The TiO₂ or ivy nanoparticle aqueous suspension was added to the DMEM solution supplemented with 10% fetal bovine serum to prepare a DMEM solution containing nanoparticles, which was used to investigate the cytotoxicity against HeLa cells. Negative controls consisted of DMEM with 10% fetal bovine serum, without the presence of nanoparticles. For apoptosis analysis, the cells were harvested 24 hours after addition of nanoparticles (1 μg/ml), fixed and stained with propidium iodide, and were then analyzed by flow cytometry using Beckman Coulter Epics XL (Beckman Coulter, Brea, Calif.) with a 488 nm argon laser.

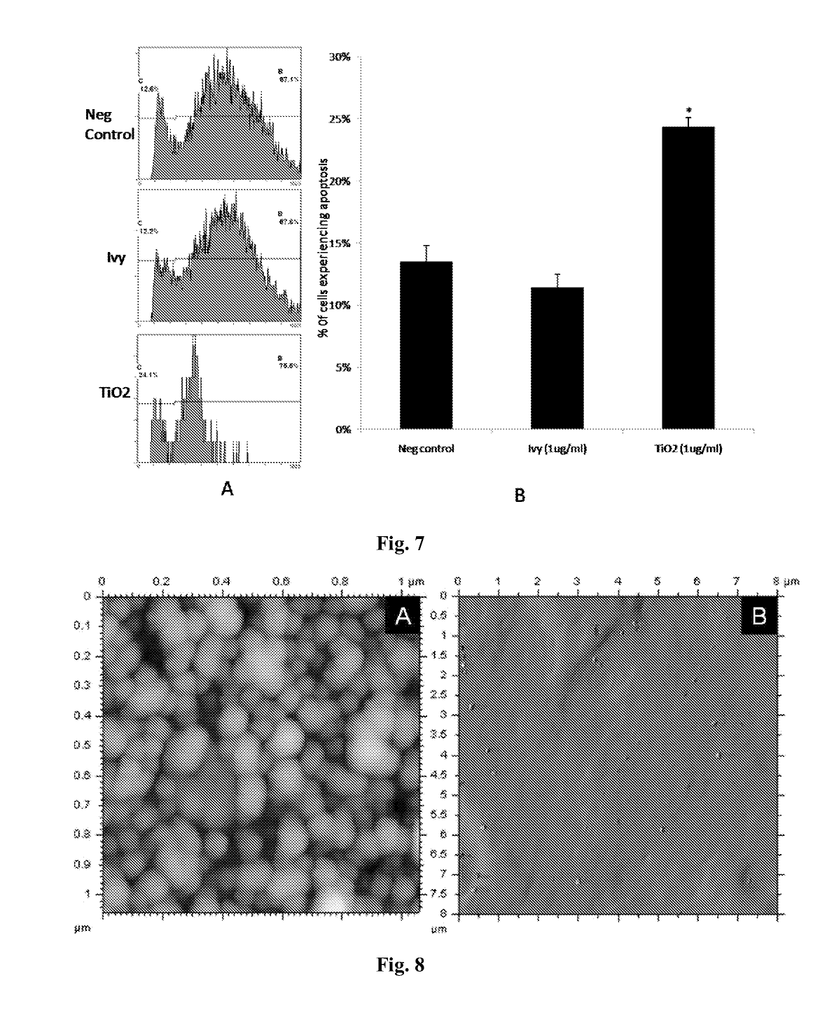

No toxicity was observed as compared to the control cells upon incubation with the ivy nanoparticles. However, in the same study, the same concentration of TiO₂ nanoparticles exhibited significant toxicity to HeLa cells. In a standard flow cytometry experiment (see FIG. 7A), Gate C in each plot was defined for the cell population with less DNA, and thus represented the cells experiencing apoptosis. Gate B from the plots was the HeLa cells with more DNA, which indicated replicating cells at differing growth stages. Statistical analysis demonstrated that there was no significant difference in the percentage of cells experiencing apoptosis between the control cell population (13.5%±1.27%) and cells incubated with ivy nanoparticles (11.5%±1.06%) (see FIG. 7B). However, in the cells incubated with TiO₂, 24.3%±0.7% of cells experienced apoptosis, which was significantly higher than the control cell population (p=0.011) and the cells incubated with ivy nanoparticles (p=0.007).

### Example 4

### Statistical Analysis

To determine if there were significant differences in cytotoxicity among HeLa cells incubated without or with different nanoparticles, a Student's t test for comparisons of each pair with 95% confidence was carried out using JMP 8 statistical software (SAS Institute Inc., Cary, N.C.).

### Example 5

### Nanoparticle Degradation

Although the cytotoxicity of ivy nanoparticles in the HeLa cell line was addressed, the possibility of these ivy nanoparticles exhibiting toxicity in the body still needed to be explored. There have been observations with gold-dendrimer nanoparticles accumulating in the liver that might damage normal liver function (Minchin, R., Nat Nanotechnol, 3:12-13 (2008)). To address this concern for ivy nanoparticles, the ability of the nanoparticles to be degraded should they pass through the skin or mucous membranes was tested. If the ivy nanoparticles were degradable, then they would be digested after their penetration through the skin and lose their normal nano-structure and, thus, any toxicity based on the nano-morphology of the particles. The degradability of nanomaterial is also beneficial to the environment when considering reports that nanomaterials have been linked to damage in fish, mortality in water fleas, and have bactericidal properties that can impact ecosystems (Oberdorster, E., 2004 and Oberdoster et al, 2005). Thus, the biodegradability of these ivy nanoparticles was also investigated in this study.

Purified nanoparticles were sonicated at 5 W for 20 minutes to physically disperse the nanoparticles in order to analyze individual particles. The experimental studies indicated that at temperatures from 4 to 37° C., the ivy nanoparticles were stable and could be readily imaged by AFM. In addition, sonication from 5-9 W was not effective at destroying the particle structure, but did serve to disperse the particles and prevent the formation of large agglomerates. Incubation of the ivy nanoparticles in RPMI, a common cell culture media, at 37° C. for up to 24 hours did not result in digestion of the nanoparticles as assessed by AFM.

To test the ability for the particles to be broken down by enzymatic digestion, sodium dodecyl sulfate (SDS) was added to the nanoparticle solution to bring the final concentration to 0.5% and 50 μg/ml of Proteinase K was added. The solution was then incubated at 37° C. from 15 minutes to 4 hours, to determine if the extent of incubation affected the digestion of the particles. Upon completion of the digestion, the sample was air-dried and imaged using AFM to determine if the nanoparticles were degraded. The control sample was prepared similarly except that the Proteinase K was not added before incubation. In addition to examining enzymatic digestion with Proteinase K, the effects of varying temperatures were examined by incubation of the nanoparticles from 4-37° C. Similarly, the nanoparticles were added to RPMI for 24 hours to determine their stability in a typical cell culture media. All samples were then air-dried and imaged by AFM.

After incubation with Proteinase K for 30 min, it was no longer possible to image the nanoparticles with AFM. As shown in FIG. 8, after enzymatic digestion, the ivy nanoparticles were degraded and lost their normal structure. This enzymatic digestion by a common proteinase could further reduce the risk to the environment and human tissues and organs. This gives organic nanoparticles a definitive advantage over metal oxide nanoparticles, since these particles resist breakdown by biological organisms and remain in the body or environment for prolonged periods of time. It was also noted that the above nanoparticles used for the Proteinase K digestion were collected based on size which also matched well with the UV 280 nm detection. As expected, they have been totally degraded. However, it is possible that there are other types or sizes of nanoparticles that had not been detected by UV detector, and as a result, were not collected for the Proteinase K digestion.

### Example 6

### Skin Penetration

Another major concern with cosmetic nanoparticles is their probability to penetrate through the skin into the circulatory system (Ryman-Rasmussen et al., Toxicol Sci, 91:159-165 (2006)). The development of proper markers for the detection of ivy nanoparticles in the skin takes considerable time; however, a mathematical modeling and computational approach can allow rapid analysis for the potential of ivy nanoparticles to penetrate though skin

Skin structure, is composed of the protective outer SC layer and a viable epidermis and dermis layer with other accessory glands (Elias, P. M., J Invest Dermatol, 125:183-200 (2005)). The penetration of particles in the skin can occur through pilosebaceous pores (diameter: 10-70 μm), sweat gland pores (diameter: 60-80 μm) and lipid matrix that fills a gap of 75 nm between dead corneocytes in the SC (see, for example, Elias, P. M. 2005; Lauer et al., Adv Drug Delivery Rev, 18:311-324 (1996); and Johnson et al., J Pharm Sci, 86:1162-1172 (1997)). As the intact skin has more than one layer in humans, ivy nanoparticles can have different diffusion activities in different layers. Ex vivo and in vivo experimental data supported that the SC has the most packaged properties and is not permeable to many chemicals and drugs (see Wartewig et al., Skin Pharmacol Physiol, 20:220-229 (2007)). A skin diffusion study indicated that the SC has a diffusion coefficient 10³ times lower than the deeper viable layer for the same chemical (Sugibayashi et al., J Control Release, 62:201-208 (1999)). Another study for nanoparticles in human skin also indicated that nanoparticles with a size of more than 20 nm rarely have a chance to penetrate through the SC layer (see Alvarez-Roman et al., J Control Release, 99:53-62 (2004)). Therefore, to understand ivy nanoparticle diffusion and penetration in human skin, it is helpful to understand the diffusion process of these nanoparticles in the SC.

There are many papers dealing with the transport of nanoparticles through the SC layer of the skin in the current literature (see, for example, Baroli et al., 2007; Zhang et al., 2008; Kohli et al., Int J Pharm, 275:13-17 (2004); Diaz-Torres et al., Appl Phys Lett, 95:043702 (2009); Kuntsche et al., Int J Pharm, 354:180-195 (2008); and Coulman et al., Int J Pharm, 366:190-200 (2009)). While the data vary depending on the experimental setup, it is generally agreed that the depth of penetration varies with the material properties of the nanoparticles, the size of individual particles, their shape, and other physicochemical factors (Kohli et al., 2004). Studies have suggested that sunscreens composed of TiO₂ and ZnO nanoparticles do not pass into the upper layers of the SC. However, as mentioned above, these studies have only examined healthy adult skin models (see Durand et al., 2009; Gontier et al., 2009; Newman et al., 2009; Oberdorster, E., 2004; and Zvyagin et al., J Biomed Opt, 13:064031 (2008)). More realistically, the skin to which the sunscreen will be applied has been damaged, either by prior sun exposure, or by a variety of other factors that damage the skin As discussed above, damage to the skin and small particle size increase the depth to which nanoparticles will penetrate (Baroli et al., 2007; Menzel et al., 2004; Larese et al., 2009; Mortensen et al., 2008; and Zhang et al., 2008).

Despite the heterogeneous structure of the SC layer, in cases where penetration is concerned, the skin behaves as a homogeneous membrane and the diffusion law still holds (see Kalia et al., Biophys J, 71:2692-2700 (1996); Kalia et al., Pharm Res, 17:1148-1150 (2000); Alberti et al., J Control Release, 71:319-327 (2001); and Schwindt et al., J Invest Dermatol, 111:385-389 (1998)). To understand the dynamic activities of the ivy nanoparticles applied to the skin, Fick's Second Law is applied and described as the follows: (∂φ/∂t)=D(∂²φ/∂χ²). The determination factor is the diffusion coefficient (D). This D is normally defined to be: D=k_{B}T/6πηR. In the case of ivy nanoparticles, R is radius of ivy nanoparticles and is set at the value of 32.65 nm, η is the viscosity of the SC lipid matrix (0.02 kg/m s), k is the Boltzmann constant (1.38×10⁻²³ m² kg s⁻² K⁻¹), and T is the absolute temperature for human skin (310.15 K). In this simplified model, the surface properties of varying nanoparticles are ignored and only the radius of the nanoparticles effects diffusion. The SC layer, which is 20 μm deep, is lipophilic and acidic with variations in gender, anatomical sites, and environmental settings (Williams, A. C., Int J Pharm, Ed. R. H. Guy, J. Hadgraft, 261:171 (2003)).

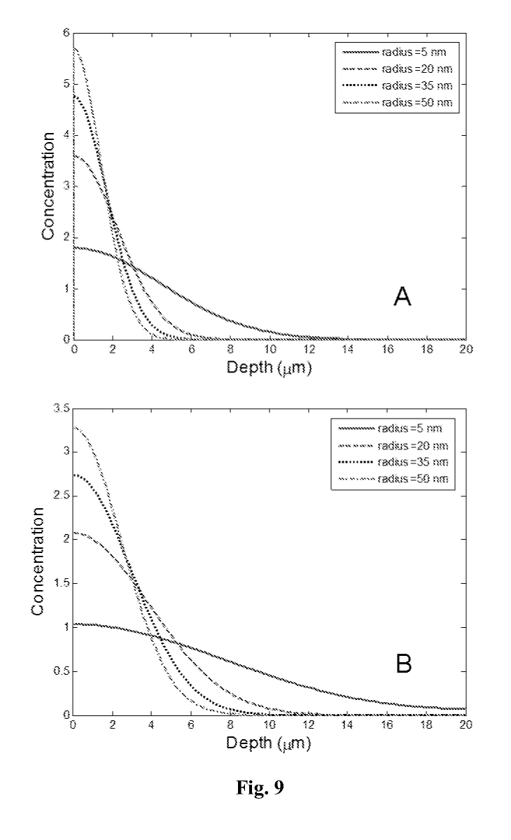

Based on the model and obtained parameters, the dynamics of nanoparticle diffusion in the SC layer of the skin was simulated. FIG. 9 shows the predicted distribution of different-sizes of nanoparticles after 8 and 20 hours of application. While nanoparticles with a diameter less than 10 nm have a chance to reach into the bottom of the SC layer (FIG. 9), nanoparticles over 40 nm can only reach 5-8 μm into the SC layer after 8 hours of application and 8-13 μm after 20 hours, as displayed in FIG. 9. This correlates with previous experimental studies about other nanoparticles within the same size range (see Popov et al., J Biomed Opt, 10:064037 (2005)). Considering that the normal period of exposure to sunlight in humans is less than 8 hours, and the diameter of the ivy nanoparticles is 65.3 nm, the ivy nanoparticles can be used in cosmetics applications without a risk of penetration.

The compounds and methods of the appended claims are not limited in scope by the specific compounds and methods described herein, which are intended as illustrations of a few aspects of the claims and any compounds and methods that are functionally equivalent are within the scope of this disclosure. Various modifications of the compounds and methods in addition to those shown and described herein are intended to fall within the scope of the appended claims. Further, while only certain representative compounds, methods, and aspects of these compounds and methods are specifically described, other compounds and methods and combinations of various features of the compounds and methods are intended to fall within the scope of the appended claims, even if not specifically recited. Thus a combination of steps, elements, components, or constituents can be explicitly mentioned herein; however, all other combinations of steps, elements, components, and constituents are included, even though not explicitly stated.

1. A cosmetic composition, comprising:
(a) a plurality of nanoparticles derived from ivy; and
(b) one or more cosmetically acceptable materials.

2. The cosmetic composition of claim 1, wherein the average diameter of the nanoparticles is from 20 nm to 100 nm.

3. The cosmetic composition of claim 1, wherein the nanoparticles comprise one or more organic compounds comprising carbon, hydrogen, and nitrogen atoms.

4. The cosmetic composition of claim 3, wherein the one or more organic compounds further comprise an oxygen atom, a chlorine atom, a sulfur atom, or mixtures thereof.

5. The cosmetic composition of claim 1, wherein the ivy is an English ivy or a Boston ivy.

6. The cosmetic composition of claim 1, wherein the one or more cosmetically acceptable materials are selected from the group consisting of organic sunscreen agents, anti-UV agents, antiperspirants, bactericides, chelating agents, colorants, complexing agents, conditioning agents, deodorants, diluents, disperants, emollients, fillers, flavorants, insect repellants, odorants, pH adjusters, plasticizers, preservatives, skin protectants, solvents, stabilizers, thickening agents, vitamins, and mixtures thereof.

7. The cosmetic composition of claim 6, wherein the anti-UV agent is a UVA filter or a UVB filter.

8. The cosmetic composition of claim 1, wherein the cosmetic composition is an emulsion.

9. The cosmetic composition of claim 8, wherein the cosmetic composition is an oil-in-water emulsion.

10. The cosmetic composition of claim 8, wherein the cosmetic composition is a water-in-oil emulsion.

11. The cosmetic composition of claim 1, wherein the cosmetic composition is prepared as a formulation for topical administration.

12. The cosmetic composition of claim 11, wherein the formulation for topical administration is a cream, an ointment, a liquid, a paste, a lotion, a foam, a gel, an emulsion, a powder, a shampoo, a conditioner, a hair rinse, a hair tonic, a hair spray, or a hair care treatment.

13. A sunscreen, comprising the composition of any of claims claim 1.

14. A plurality of nanoparticles, the particles each comprising:
an organic compound comprising at least 60% of the chemical composition of the nanoparticles,
wherein the nanoparticles are derived from ivy.

15. The nanoparticles of claim 14, wherein the average diameter of the nanoparticles is from 20 nm to 100 nm.

16. The nanoparticles of claim 14, wherein the organic compound comprises carbon, hydrogen, and nitrogen atoms.

17. The nanoparticles of claim 16, wherein the organic compound further comprises an oxygen atom, a chlorine atom, a sulfur atom, or mixtures thereof.

18. The nanoparticles of 14, wherein the ivy is an English ivy or a Boston ivy.

19. A method of preparing a cosmetic composition, comprising mixing a plurality of nanoparticles derived from ivy with one or more cosmetically acceptable materials.

20. The method of claim 19, further comprising isolating the nanoparticles from the ivy prior to mixing the nanoparticles with one or more cosmetically acceptable materials.

21. The method of claim 20, further comprising purifying the nanoparticles prior to mixing the nanoparticles with the one or more cosmetically acceptable materials.

22. The method of claim 21, further comprising treating the cosmetic composition with an emulsifier to provide an emulsified cosmetic composition.

23. The method of claim 19, further comprising formulating the cosmetic composition for topical administration.

24. The method of claim 23, wherein the cosmetic composition is formulated for topical administration as an ointment, a liquid, a paste, a cream, a lotion, a foam, a gel, an emulsion, a powder, a shampoo, a conditioner, a hair rinse, a hair tonic, a hair spray, or a hair care treatment.

25-32. (canceled)
